(19) United States
(12) Patent Application Publication
(10) Pub. No.: US 2021/0335484 A1
(43) Pub. Date: Oct. 28, 2021

(54) MEDICAL PRESCRIPTIONS OPTIMIZED VIA PAYMENT TRANSACTION NETWORK

(71) Applicant: MASTERCARD INTERNATIONAL INCORPORATED, Purchase, NY (US)

(72) Inventors: Marek KURYLKO, Bloomfield, NJ (US); Eugene REDA, Little Falls, NJ (US); Joseph HAYES, Montclair, NJ (US)

(21) Appl. No.: 16/860,728

(22) Filed: Apr. 28, 2020

(51) Int. Cl. G16H 40/20; G16H 10/60; G16H 20/10; G06Q 20/02; G06Q 30/02; G06Q 40/08; G06Q 10/10; G06Q 40/02; G06Q 20/40; G06F 3/0482

(52) U.S. Cl. CPC G16H 40/20; G16H 10/60; G16H 20/10; G06Q 20/027; G06F 3/0482; G06Q 40/08; G06Q 10/10; G06Q 40/02; G06Q 20/40; G06Q 30/0283

(57) ABSTRACT

A computing device executing a payment application with prescription feature can initiate an optimized prescription fill request of a medical prescription via a payment network by at least transmitting user information including a name of a patient to a payment gateway such as a point of sale terminal or online payment gateway; receive pricing options for the medical prescription from a server supporting optimized prescription filling; obtain pharmacy information including names and/or addresses of at least one pharmacy that can fill the medical prescription; provide the pricing options for the medical prescription via a user interface; provide the pharmacy information via the user interface; receive a selection of a pharmacy of the at least one pharmacy and a payment indication; and transmit the selection of the pharmacy and the payment indication to the payment gateway.

### BACKGROUND

Consumers generally do not enjoy filling a prescription at a pharmacy. Filling a prescription often involves waiting in a line to provide the prescription to a limited number of pharmacists, waiting for one of the pharmacists to fill the prescription, and waiting to speak with the pharmacist to dispense the medicine and provide any counseling. Further delays can result from the pharmacy contacting a consumer's insurance provider to receive pricing and/or authorization from the provider and from paying for the medicine. These delays can be uncomfortable for unhealthy individuals or for parents of unhealthy children.

In addition, a consumer's price expectation for a medicine is not always met by a particular manufacturer, pharmacy, or the consumer's insurance. Such a situation can occur when a consumer changes insurance, for example, due to changing employers.

Thus, a consumer might be unable to afford the medicine or otherwise uninterested in paying for the medicine. Such an outcome results in time wasted in awaiting fulfillment of the prescription or in wasting additional time in awaiting fulfillment of an alternative (e.g., generic) version of the prescription.

### BRIEF SUMMARY

Payment transaction networks can be used to optimize prescription filling. Aspects of the present disclosure use a payment application (e.g., a built-in wallet application for a smartphone, a card issuer's smartphone application, or a merchant application with payment capabilities) to connect to a payment gateway, e.g., via a point-of-sale (POS) terminal at a doctor's office. Thus, the application can leverage the payment transaction network to simplify a consumer's prescription-filling experiences.

A computing device executing a payment application with a prescription feature can initiate an optimized prescription fill request of a medical prescription via a payment network by at least transmitting user information including a name of a patient to a payment gateway such as a point of sale terminal or online payment gateway; receive pricing options for the medical prescription; obtain pharmacy information including names and/or addresses of at least one pharmacy that can fill the medical prescription; provide the pricing options for the medical prescription via a user interface; provide the pharmacy information via the user interface; receive a selection of a pharmacy of the at least one pharmacy and a payment indication; and transmit the selection of the pharmacy and the payment indication to the payment gateway.

A server supporting optimized prescription filling can receive a request for an optimized prescription fill, the request comprising user information, including a name of a patient, and a prescription for a medicine for the patient. In response to receiving the request, the server can obtain medication information at least including pricing information for the prescription for the medicine for the patient; and can transmit the medication information to a payment application with prescription feature. The server can receive a selection of a selected pharmacy and a payment indication; and transmit an authorization of a dispensation of the medicine to the patient for the selected pharmacy along with the payment indication. The authorization of dispensation can be sent via an insurance provider system.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates an example operating environment to which the present disclosure for optimizing fulfillment of medical prescriptions via a payment network can be applied.

FIGS. 2A and 2B illustrate a flow diagram for an implementation of prescription optimization via a payment network.

FIGS. 3A and 3B illustrate a flow diagram for a merchant-of-record implementation of prescription optimization via a payment network.

FIG. 4 illustrates an example method for a prescription feature.

FIGS. 5A-5C illustrate representational graphical user interfaces of a payment application with prescription feature.

FIG. 6 illustrates an example method for optimized prescription filling.

FIG. 7A illustrates example workflow of a computing device performing the method illustrated in FIG. 4.

FIG. 7B illustrates an example workflow of a prescription optimization service performing the method illustrated in FIG. 6.

FIG. 8 illustrates components of an example computing device that may be used by a consumer for optimizing prescription filling.

FIG. 9 illustrates components of an example computing system that may be used for prescription optimization.

### DETAILED DESCRIPTION

Payment transaction networks can be used to optimize prescription filling. Aspects of the present disclosure use a payment application (e.g., a built-in wallet application for a smartphone, a card issuer's smartphone application, or a merchant application with payment capabilities) to connect to a payment gateway, e.g., via a point-of-sale (POS) terminal at a doctor's office. Thus, the application can leverage the payment transaction network to simplify a consumer's prescription-filling experiences.

In one example implementation of the payment application, an issuer's banking application on a user's smartphone is used to improve the experience of a consumer (e.g., a patient or a patient-beneficiary) in filling a prescription for a medication or treatment. For example, the application can inform the consumer, in advance of going to a pharmacy, whether there is a generic available for the medication or treatment and what its cost will be. The application can route the prescription to a pharmacy other than the consumer's default in the doctor system, for example, on late night trips at an urgent care facility.

The application additionally can provide functionality for the consumer to prepay for the medication or treatment or to pay on location at the pharmacy. Thus, the application can improve the consumer's experience at the pharmacy by enabling prepayment for the medication or treatment and permitting the consumer to “beat the line.” In addition, the application can permit the consumer to avoid the shock of getting through the pharmacy line, only to find out that the medication or treatment is more expensive than expected.

Although this disclosure uses the term “medicine” as the subject of a prescription, this disclosure also contemplates that a prescription can be for a treatment, as well.

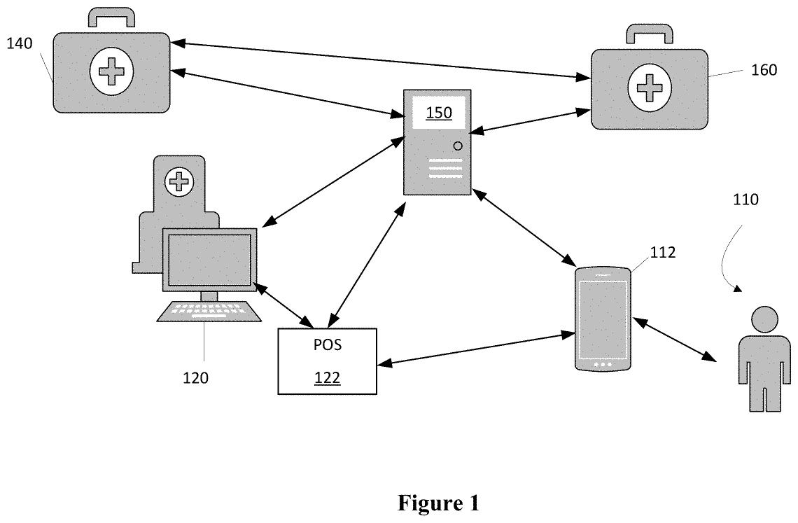

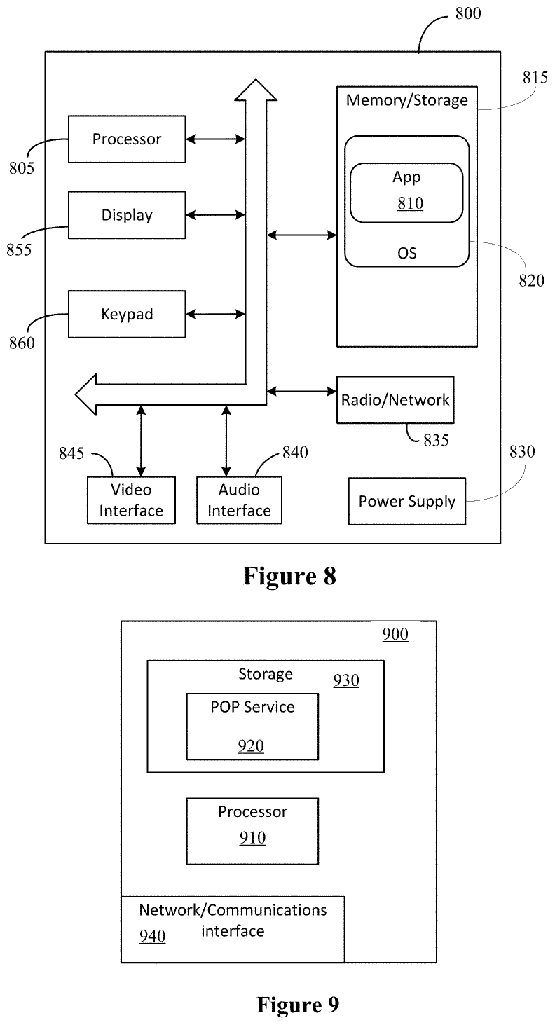

FIG. 1 illustrates an example operating environment to which the present disclosure for optimizing fulfillment of medical prescriptions via a payment network can be applied. Referring to FIG. 1, an operating environment can include a consumer 110 with a computing device 112, a doctor system 120 and point of sale (POS) terminal 122, an insurance service 140 operated by a health insurance carrier of the consumer 110, a prescription optimization (POP) service 150 that routes transaction information, and a pharmacy 160. Computing device 112 may be embodied as described with respect to computing device 800 of FIG. 8. POP service 150 may be implemented via a computing system embodied as described with respect to computing system 900 of FIG. 9.

In a use scenario, the consumer 110 visits the doctor's office to receive medical attention. The doctor prescribes a medicine that is entered into the doctor system 120. Then, when the consumer 110 is ready to check out, the consumer 110 can be informed that there is a prescription and that the consumer 110 can use the payment network to select and optionally prepay for the prescription. The consumer 110 can use an application on the computing device 112 to effectuate the optimized fulfillment of the prescription. For example, the doctor system 120 transmits information regarding doctor services and the medicine to doctor payment gateway 122 (e.g., doctor point of sale (POS) terminal). The doctor POS terminal 122 can receive from the consumer 110, via the application on the computing device 112, payment card information as well as other user information. In some cases, the action to effectuate the communication between computing device 112 and POS terminal 122 is in the form of a contactless payment or a “tap” to pay. The doctor system 120—either directly or via the POS terminal 122—transmits the information received via the POS terminal 122 to the POP service 150. The POP service 150 receives the information from the doctor system 120 (or POS terminal 122). The POP service 150 may be implemented on a system in a payment network or on a system in communication with the payment network.

In some cases, the POP service 150 interfaces with the insurance service 140 to obtain selection information for the consumer 110 regarding the medicine and, in some cases, pharmacies. The POP service 150 can transmit the selection information (e.g., medication information from the insurance service) to the computing device 112 for selection by the consumer 110. The selection information can include the medicine options (which can include alternatives), images of the medicine (and alternatives), and expected pricing of the medicine (and alternatives). The information can be presented via the application at the computing device 112. For example, a user interface of the application at the computing device 112 may update to reflect the prescription, image, price, and even whether a generic is available. In addition to selecting prescription brand or generic, the application can enable the consumer 110 to select a pharmacy and whether to prepay for the prescription. Once the consumer makes their selections, the computing device 112 can transmit the selections as confirmed information to the doctor POS terminal 122, which relays the confirmed information, including payment/prepayment indication (e.g., whether the consumer selected prepayment) to the POP service 150. By routing the confirmed information through the doctor, no medical information is stored by the issuer or other entity (which may manage the application).

In some cases, the POP service 150 transmits some or all of the confirmed information to the insurance service 140. The insurance service 140 then transmits an authorization to the pharmacy 160. In some cases, the POP service 150 transmits the confirmed information to the pharmacy 160.

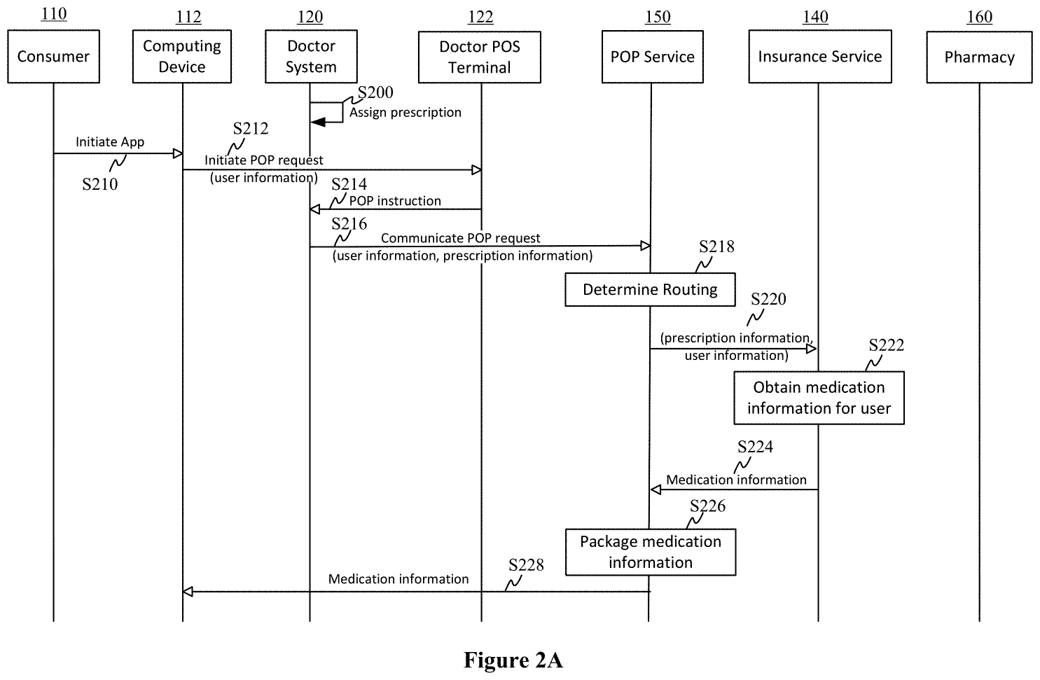

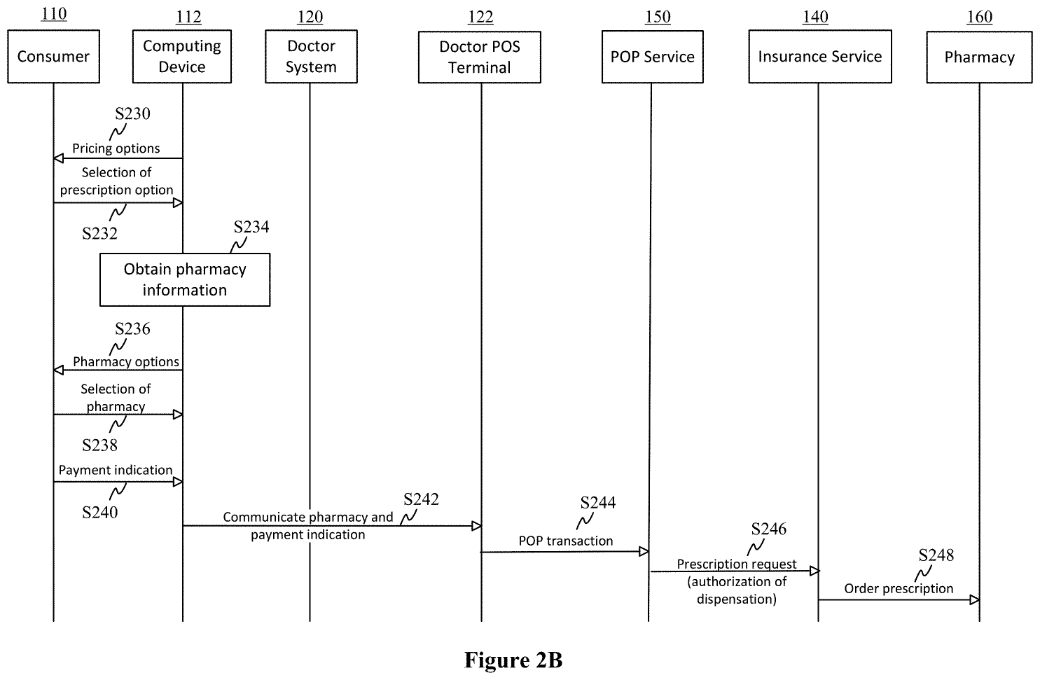

FIGS. 2A and 2B illustrate a flow diagram for an implementation of prescription optimization via a payment network. In the example implementation illustrated in FIGS. 2A and 2B, the application at the computing device 112 is configured as a wallet application with a prescription optimization feature. As a preliminary step, a medical professional assigns (S200) a prescription to the consumer. The prescription may be for a medicine, such as a prescription drug or an over the counter drug, and/or a medical implement (e.g., glucose test strips, needles and syringes).

Once the prescription is in the doctor system 120, the prescription can be subject to the described optimized prescription filling. For example, the doctor system 120 can transmit transaction information to the doctor POS terminal 122 to set up the terminal for a prescription optimization (POP) process. In one implementation, the transaction can also include a fee for the doctor's appointment. Then, when the consumer 110 is ready to check out after their visit with the medical professional, the consumer 110 can initiate (S210) their application with optimized prescription filling feature on the computing device 112. As previously mentioned, the optimized prescription filling feature can be part of an issuer's banking application or even part of a stand-alone wallet application.

The consumer 110, via the application on the computing device 112, can initiate a communication between the computing device 112 and the doctor POS terminal 122 in order to initiate (S212) an optimized prescription fill request (referred to here as a “POP request”) via a payment network. The communication between the computing device 112 and the doctor POS terminal 122 includes user information for the POP request and can be accomplished via any suitable communication between a computing device with an application supporting “contactless” payments and a POS terminal.

Some implementations of communication between the computing device 112 and the doctor's POS terminal 122 are performed via Wi-Fi. Wi-Fi is a group of wireless communication technologies, based on the Institute of Electrical and Electronics Engineers (IEEE) 802.11 family of standards. Other implementations perform the wireless communication with Bluetooth. Still other implementations of wireless communication between the computing device use contactless technologies, such as Near-Field Communication (NFC) technologies. Although NFC does not require contact between the computing device 112 and the doctor POS terminal 122, many use cases involve physical contact between the computing device 112 and the doctor POS terminal 122 as a matter of convenience. For example, when using NFC, the consumer may tap the computing device 112 to the doctor POS terminal 122.

Thus, in one implementation, the computing device 112 transmits, as part of initiating the POP request (S212), user information identifying the patient (whether the consumer or beneficiary thereof) to the doctor POS terminal 122. The user information can include, for example, a name or a group and member number with an insurance carrier. Doctor POS terminal 122 then communicates (S214) the user information to the doctor system 120 as part of a POP instruction to the doctor system 120. The POP instruction indicates to the doctor system 120 that a consumer 110 has initiated a POP request. In some cases, as an alternative, some or all of the user information may be manually entered to the doctor system 120.

The doctor system 120 then communicates (S216) a POP request to a sever supporting optimized prescription filling via a POP service 150. The POP request can be made via an application programming interface (API) provided by POP service 150. The POP request can include the user information and the prescription information. The user information can include, for example, one or more of a name of the patient, location information of the user, insurance information of the user, and any preferences of the user (e.g., regarding prescriptions and/or pharmacies). The prescription information can include a name of the prescribed medicine, an identification of the prescribed dosage of the medication, and a quantity of the medicine to be dispensed.

The POP service receives the user information, which includes at least a name of the patient, and the prescription information indicating the prescription for a medicine for the patient. The POP service 150 determines (S218) the appropriate wallet routing of the prescription (e.g., which application that the medication information should be sent to). The routing information may be obtained from a storage resource storing registration information of the consumer when they set up the application with prescription optimization feature.

In some cases, the POP service 150 communicates with an appropriate insurance carrier for the medication information for consumer via, for example, an insurance service 140 that may be available from that appropriate insurance carrier. The insurance service 140 can be provided by an insurance provider to enable access to information on the consumer's insurance plan and/or available coverage regarding the prescribed medicine.

For example, the POP service can transmit (S220) the prescription information and the user information to an insurance service 140. The insurance service 140 can obtain (S222) the appropriate medication information for the consumer based on the consumer's insurance policy; and provide (S224) relevant details (e.g., pricing of brand name and generics) to the POP service 150. In some cases, the insurance service 140 can indicate whether the prescribed medicine is covered by the consumer's insurance policy and/or what alternatives of the medicine are covered. The insurance service may additionally indicate an anticipated price for the medication and any identified alternatives at the prescribed dosage based on the coverage of the insurance plan for the patient.

The anticipated price can be influenced in many ways, such as volume or exclusivity discounts from a manufacturer of the medication at the prescribed dosage. For example, the insurance carrier can sign a manufacturer-specific business deal in which the manufacturer (especially a generic manufacturer) reduces a price for the medication at the prescribed dosage in return for favorable treatment of the manufacturer, relative to the manufacturer's competitors, by the insurance carrier. The insurance carrier can sign a pharmacy-specific business deal in which a pharmacy (e.g., national or regional pharmacy chain) reduces a price for the medication at the prescribed dosage for favorable treatment of the pharmacy, relative to the pharmacy's competitors, by the insurance carrier. A manufacturer (especially a generic manufacturer) can sign a business deal with a pharmacy (e.g., national or regional pharmacy chain) to reduce a price for the medication at the prescribed dosage for favorable treatment of either entity, relative to its competitors, by the other entity. When the insurance carrier has a pharmacy-specific pricing, the insurance service 140 can provide the pharmacy information with the medication information in step S224.

The POP service 150 can package (S226) the medication information for the consumer's prescription and communicate (S228) the medication information to the computing device 112. Images of the medication (e.g., packaging or the medication itself) may be obtained from the insurance service 140 as part of the medication information or from publicly available resources such as accessed via a search engine (e.g., via an image search).

The POP service can transmit the package of medication information (in communication S228) using, for example, a push notification via an API through the application with prescription feature executing on the computing device 112. In some cases, the POP service may use other communication channels such as short message service (SMS) or multimedia message service (MIMS).

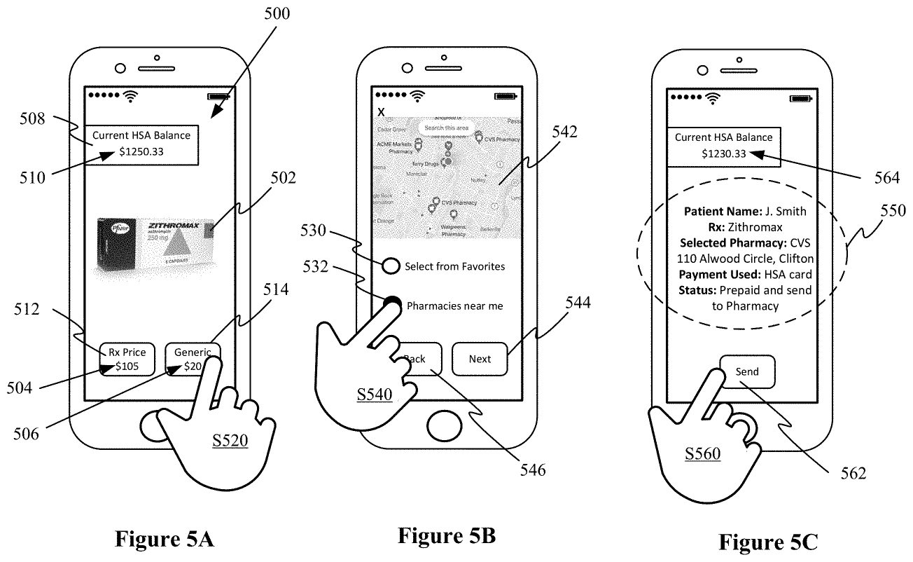

Referring to FIG. 2B, when the computing device 112 receives the medication information, including pricing options for a medical prescription, from the POP service 150, the computing device 112 can provide (S230) the pricing options for the medical prescription via a user interface of the application. FIG. 5A illustrates an example user interface with pricing option view.

The computing device 112 can receive (S232) from the consumer 110 an input selecting a particular medical prescription option (e.g., brand, generic).

The computing device 112 can obtain (S234) pharmacy information such as names and/or addresses of at least one pharmacy that can fill the medical prescription; and provide (S236) the pharmacy information as options to the consumer 110. The pharmacy information can further include hours of operation, and whether the pharmacies allow for prepayment of the prescription or not. FIG. 5B illustrates an example user interface for initiating a pharmacy search.

When obtaining pharmacy information in operation S234, the computing device can determine its geolocation, for example, using an internal sensor. In one implementation, the computing device 112 retrieves its Global Positioning System (GPS) coordinates. Alternatives for geolocation determination technology include GLONASS (Globalnaya Navigatsionnaya Sputnikovaya Sistema), DORIS (Doppler Orbitography and Radiopositioning Integrated by Satellite), BeiDou/COMPASS, Galileo, IRNSS (Indian Regional Navigation Satellite System), and QZSS (Quasi-Zenith Satellite System). Additional alternatives include cellular and Wi-Fi positioning. The computing device 112 can then communicate the geolocation (e.g., with search term pharmacy) to a map service such as GOOGLE maps or BING maps to request a map and nearby pharmacies. In some cases, the computing device 112 may transmit the geolocation to either the insurance service 140 or the POP service 150. The insurance service 140 or the POP service 150 then transmits the local pharmacies to the computing device with or without additional filtering such as for indicating those that accept the consumer's insurance carrier.

In some cases, when providing the pharmacy information as options in operation S236, the computing device 112 filters the pharmacies that are presented to the consumer to only those previously selected by the consumer as a favorite. In addition, the computing device can filter the pharmacies to only those that meet the prescription option selected by the consumer.

In some cases, the computing device 112 retrieves the current geolocation of the computing device using, e.g., a GPS receiver. Accordingly, the computing device 112 can also filter the pharmacies to only those within a predetermined distance, e.g., 5 miles, of the current location of the computing device 112.

In some cases, the computing device 112 compares the hours of availability of each of the pharmacies against the current time to determine which of the pharmacies is currently available to fill the prescription. The computing device 112 then filters the pharmacies to only those that are currently available to fill the prescription.

In a further implementation, the computing device filters the pharmacies to only those that allow for prepayment.

The computing device 112 can receive (S238) from the consumer 110 an input selecting one of the pharmacies. The interface of the application can include options for the consumer to pre-pay for the medication or to pay at the selected pharmacy. The computing device 112 can thus receive (S240) a payment indication from the consumer that indicates whether the consumer wants to pre-pay for the medication or to pay at the selected pharmacy.

If the consumer selects to pre-pay for the medication, the computing device 112, via the application, can notify the consumer of any bank, credit, or gift accounts linked to the application and even present the consumer with an option to add a new or transaction-specific payment method. The new or transaction-specific payment method can be any electronic payment method, such as a credit card, debit card, gift card, bank account, payment service (e.g., PayPal), HSA, and so on. In some implementations, the application saves the new or transaction-specific payment method for later use.

In some cases, when the consumer selects to pre-pay for the medication, the computing device 112 receives a selection of a linked account or a new or transaction-specific payment method as part of the payment indication.

Upon completing the selections, the application transmits the selection of the pharmacy and the payment indication to the payment gateway by, for example, communicating (S242) with the doctor POS terminal 122. The doctor POS terminal 122 sends (S244) the transaction information (e.g., a POP transaction) to the POP service 150.

The POP service 150 receives the POP transaction, which includes the selection of a selected pharmacy and the payment indication, and transmits a prescription request (S246), which provides an authorization of a dispensation of the medicine to the patient for the selected pharmacy along with the payment indication. The authorization of dispensation can be received by the insurance service 140, which can then transmit (S248) the prescription order to the selected pharmacy. The prescription order provides an authorization to the pharmacy to dispense the medication. The prescription order can include the name of the patient, the name of the medication, and the quantity of the medication. In some implementations, as an alternative, the POP service 150 communicates the prescription request to the doctor system 120, which can transmit the prescription order to the selected pharmacy.

The selected pharmacy 160 receives the name of the patient, the name of the medication, and the quantity of the medication. The selected pharmacy 160 can also receive the indication as to whether the consumer has prepaid for the prescription. The selected pharmacy can then begin preparing the medication.

Accordingly, if the consumer prepaid for the medicine, the consumer can simply pick up the medicine from the pharmacy (and pharmacist) directly or from a pickup location such as a locker (e.g., a prepaid purchase locker). They consumer may be required to present identification or have a code to access the locker. The consumer does not need to wait to present the prescription, consider the cost and/or availability of alternatives, wait for the medicine to be dispensed, wait for a financial transaction, and/or wait in a line to do those acts. If the consumer did not prepay for the medicine, the consumer can purchase the medicine according to conventional methods at the pharmacy.

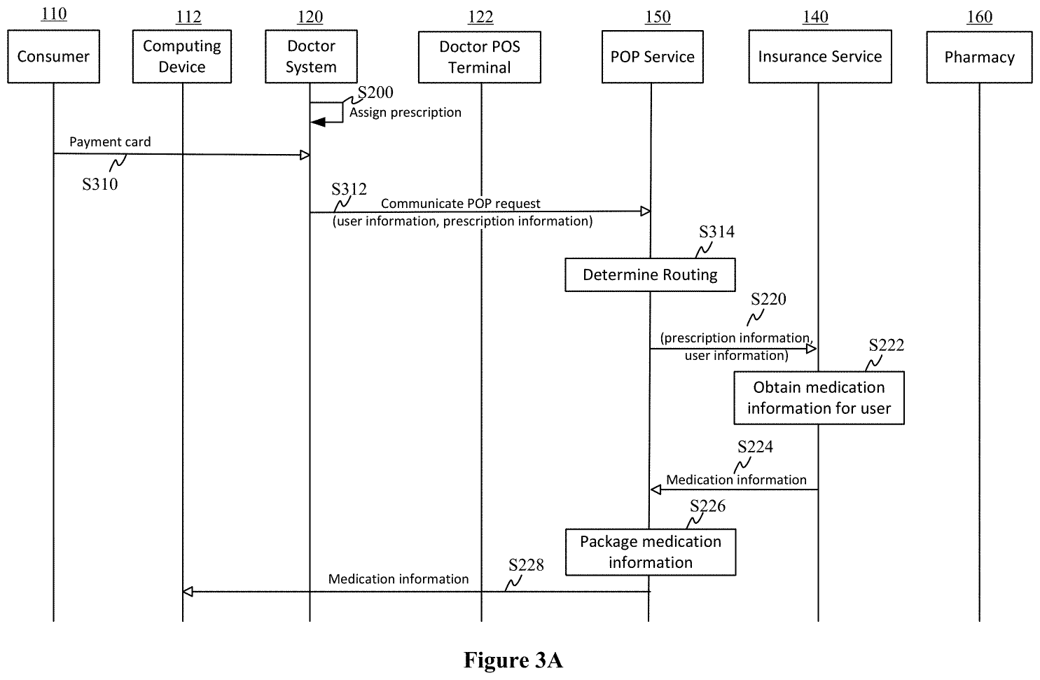

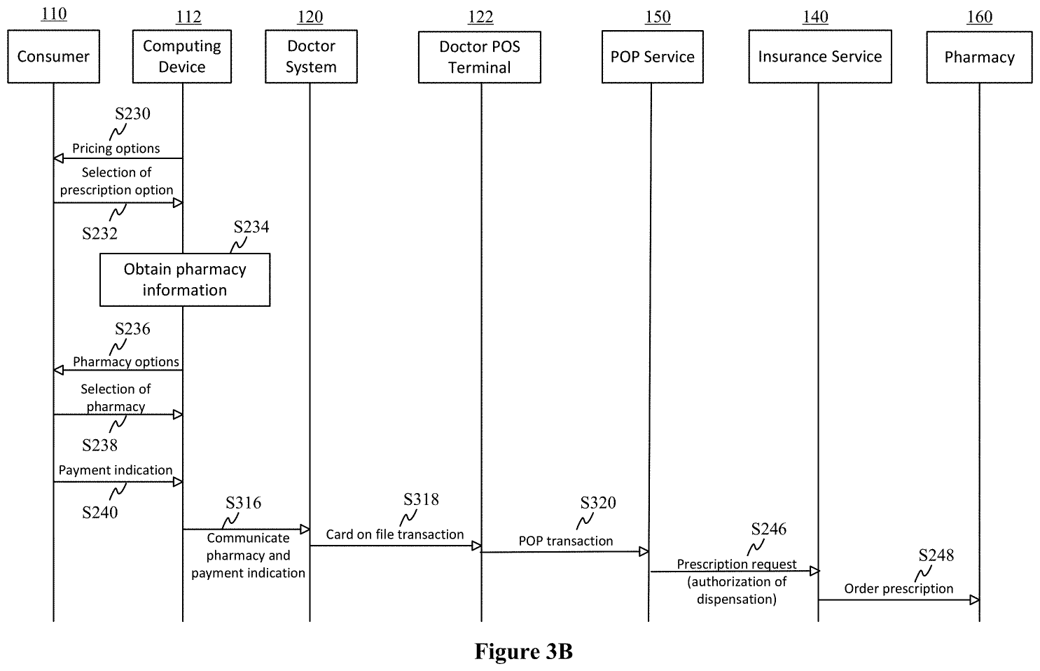

FIGS. 3A and 3B illustrate a flow diagram for a merchant-of-record implementation of prescription optimization via a payment network. In the merchant-of-record scenario, the consumer does not initiate the POP request via an application with prescription feature. This implementation is suitable for scenarios where a doctor's POS terminal does not support contactless payments.

An example consumer experience begins similarly as that described with respect to FIG. 2A with a medical professional assigning (S200) a prescription to their patient (e.g., consumer or beneficiary thereof). Instead of the consumer initiating their application with optimized prescription filling feature as described with operation S210 of FIG. 2A, in the merchant-of-record scenario, payment information of the consumer's payment card is received (S310) at the doctor's system. The payment information may be provided to the doctor's system 120 at any time before a POP request is made (and may be manually entered into the doctor's system). For example, a funding primary account number (the payment information) may be kept on file at the doctor's computing system 120 and can be used upon the request of the consumer 110 to initiate prescription optimization.

The doctor system then communicates (S312) a POP request to a sever supporting optimized prescription filling via a POP service 150. The POP request can be made via an application programming interface (API) provided by POP service 150. The POP request can include the user information and the prescription information.

The POP service receives the user information, which includes at least a name of the patient, and the prescription information indicating the prescription for a medicine for the patient. The POP service 150 determines (S314) the appropriate wallet routing of the prescription (e.g., which application that the medication information should be sent to). The routing information may be obtained from a storage resource storing registration information of the consumer when they set up the application with prescription optimization feature. The routing information can be associated with the consumer's FPAN received as part of the user information with the POP request.

Operations S220, S222, S224, S226, S228, S230, S232, S234, S236, S238, and S240 can be as described with respect to FIGS. 2A and 2B.

In the merchant-of-record scenario, when the consumer 110 finishes providing pharmacy selection and payment indication, the computing device 112 communicates (S316) the pharmacy and payment indication to the doctor system 120, which performs a card-on-file transaction (S318) that can use the POS terminal 122 to request (S320) a POP transaction. The communication of operation S316 can be carried out via an API of the software at the doctor's system 120. Accordingly, in this implementation, the application at the consumer's computing device 112 does not have to include a wallet function. Rather, if the consumer 110 selected to prepay for the medication, the doctor system 120 can use the previously entered FPAN to pay for the medicine. Other implementations, such as paying from the computing device or accepting a new FPAN for prepayment of the medicine, are also possible.

Operations S246 and S248 can then be performed as described with respect to FIG. 2B.

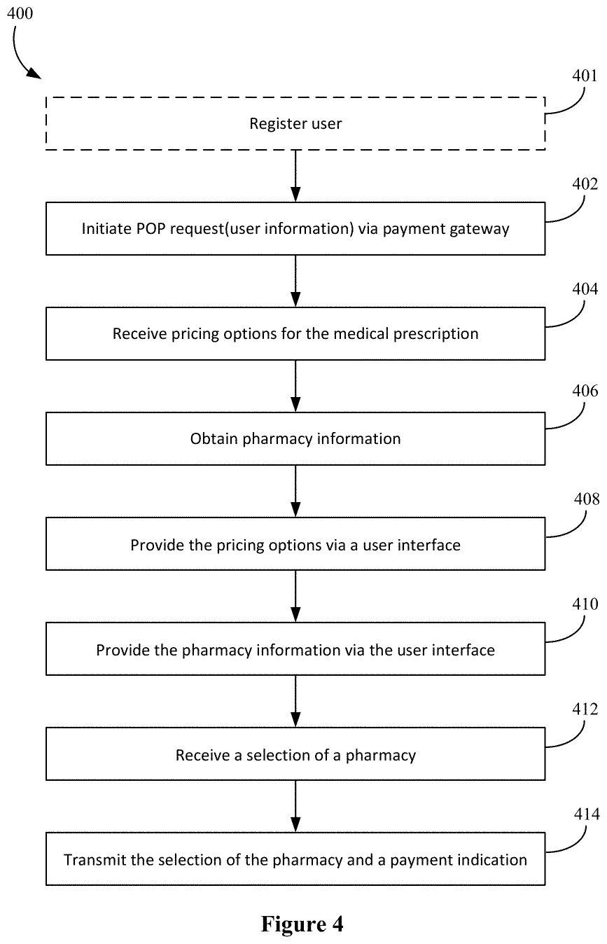

FIG. 4 illustrates an example method for a prescription feature. A payment application with prescription feature, such as described with respect to FIGS. 2A and 2B can include instructions that direct a computing system, such as computing system 112, to perform method 400. As mentioned briefly above, a consumer may register (401) with the POP service to enable prescription optimization. In some cases, the registration process may be carried out via a participating mobile application. For example, a consumer may select to enroll in the POP feature of an issuer application. When enrolling in an application with prescription feature, the consumer would accept the terms and conditions and enter their medical insurance details (e.g., carrier, policy number, users). Particular implementations to which the present disclosure can be applied include gathering, use, and disclosure of data from various sources to improve quality and experience. The present disclosure contemplates that, in some instances, this gathered data may include personal information, such as health information. The present advancement contemplates that the entities involved with such personal information respect and value privacy policies and practices. Further, any personal information and/or health information would be handled according to proper regulations.

After evaluating the terms and conditions, the consumer can accept the terms and conditions through an input (e.g., click or tap) to the computing device. By permitting the consumer to consider and accept the terms and conditions, entities involved with use and disclosure of a consumer's personal information (including health information) can comply with the Health Insurance Portability and Accountability Act (1996) (“HIPAA”). In some cases, based on GPS, the consumer is asked by the application to select their favorite local pharmacies and to indicate which payment methods they want to use with the service.

Then, for use of the prescription feature, method 400 can include initiating (402) an optimized prescription fill request of a medical prescription. The initiation of the optimized prescription fill request can include communicating user information via a payment gateway. This step may be omitted for the merchant-of-record scenario or for implementations where the application does not also include a wallet feature. The method 400 further includes receiving (404) pricing options for the medical prescription, for example, from a POP service; obtaining (406) pharmacy information including names and/or addresses of at least one pharmacy that can fill the medical prescription; providing (408) the pricing options for the medical prescription via a user interface; providing (410) the pharmacy information via the user interface; receiving (412) a selection of a pharmacy of the at least one pharmacy and a payment indication; and transmitting (414) the selection of the pharmacy and the payment indication to the payment gateway.

### Enrollment Process:

FIGS. 5A-5C illustrate representational graphical user interfaces of a payment application with prescription feature. Referring to FIG. 5A, the application can provide a pricing option view 500. Here, an image of the prescription 502, the brand price 504, and the generic price 506 are shown. In some cases, such as illustrated in the example, a funding source 508 and balance 510 may be displayed as part of the pricing option view 500, which may be used to inform the consumer when the consumer makes their decision. In the illustrated example, the funding source 508 is a health savings account (HSA). The consumer may select the brand or the generic version via selection icons 512, 514, respectively. Here, the consumer selects (S520) the generic version via the associated icon 514.

Referring to FIG. 5B, the application can enable a user to initiate a pharmacy search for identifying a pharmacy to direct the prescription. The consumer may, for example have the option to select from their favorite pharmacies (e.g., via a first selection icon 530) or to select from pharmacies nearby (e.g., via a second selection icon 532). In the illustrative example, the consumer selects (S540) the option “pharmacies near me” (via the second selection icon 532) to search for pharmacies within a certain radius around the coordinates of the location of the consumer's computing device. The list of pharmacies may alternatively be based on an IP address of the computing device or the address of the consumer. The application can use the location information to obtain a map view 542 with the consumer's position and nearby pharmacies, for example via available application programming interfaces with a map service such as GOOGLE maps. Also shown in the example illustration are icons 544, 546 that respectively enable the consumer to proceed to the next screen or revert back to the previous screen.

Referring to FIG. 5C, the application can provide a pre-confirmation page. In some implementations, the pre-confirmation preview of information 550 includes the name of the patient, whether the consumer's preferred a brand name or generic alternative, the name and/or address of the selected pharmacy, and whether the user selected to pre-pay for the medicine. Other information, such as payment source (e.g., credit card and credit card number), hours of availability of the selected pharmacy, price expectation, and status of prescription, can be provided as part of the pre-confirmation page. In some implementations, the pre-confirmation also includes the name of the medicine and an image of the selected medication. In the illustrated example, the consumer confirms (S560) the information via a send icon 562. In the illustrated scenario, because the user selected the generic, which was indicated to cost $20, the balance 564 is updated to reflect the deduction of the cost of the generic. The information selected by the consumer can be transmitted to the POP service to effectuate the optimized prescription filling.

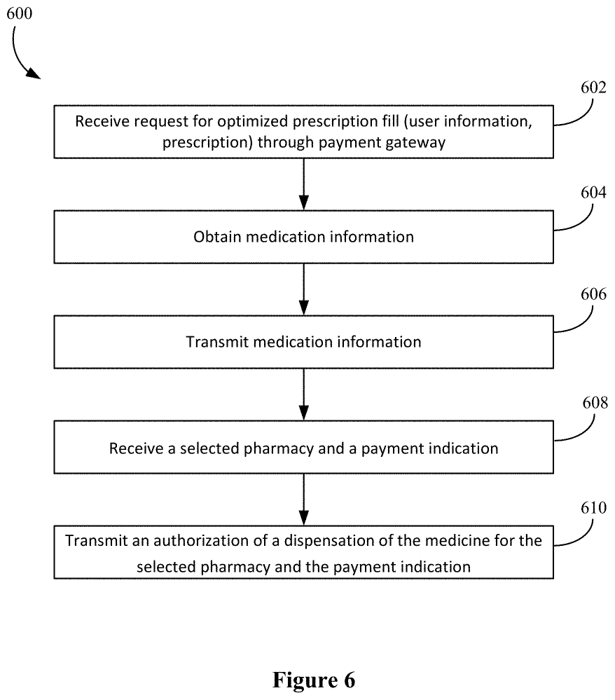

FIG. 6 illustrates an example method for optimized prescription filling. As described with respect to FIGS. 2A and 2B (and FIGS. 3A and 3B), a POP service can include instructions that direct a server supporting optimized prescription filling, such as computing system implementing POP service 150, to perform method 600. Method 600 can include receiving (602) a request for an optimized prescription fill through a payment gateway, where the request includes user information including a name of a patient and a prescription for a medicine for the patient. In response to receiving the request, the method 600 can further include obtaining (604) medication information at least including pricing information for the prescription for the medicine for the patient; and transmitting (606) the medication information to a payment application with prescription feature. The method 600 further includes receiving (608) a selection of a selected pharmacy and a payment indication; and transmitting (610) an authorization of a dispensation of the medicine to the patient for the selected pharmacy along with the payment indication. The authorization of dispensation can be sent via an insurance provider system.

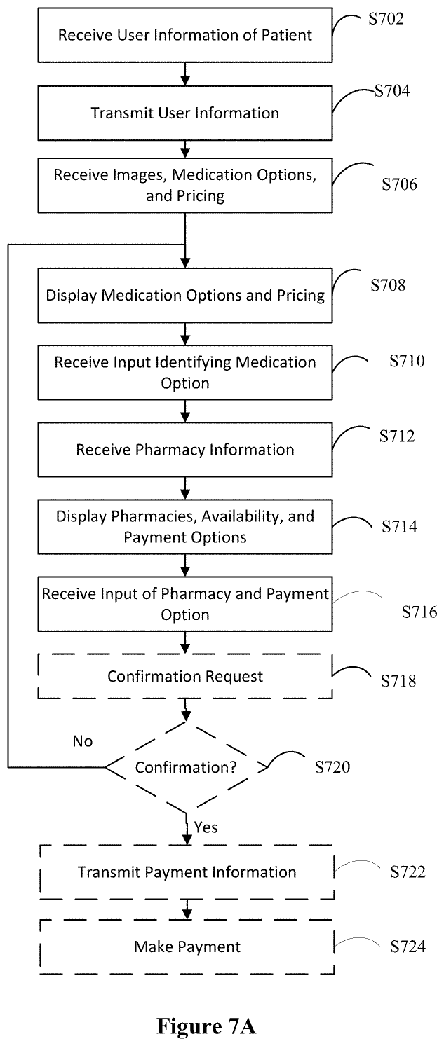

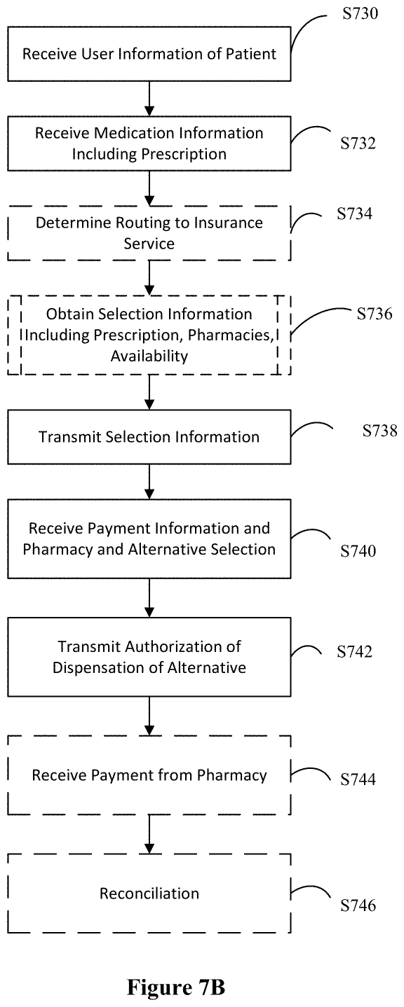

FIG. 7A illustrates an example workflow of a computing device performing the method illustrated in FIG. 4. Referring to the workflow of FIG. 7A, a computing device receives (S702) user information. The user information can include a name of the consumer, a name or other identifier of the insurance carrier, a policy number for the consumer and/or a group number for the consumer, and names of any beneficiaries of consumer.

The computing device then transmits (S704) the user information. In implementations in which the doctor office includes a POS terminal, the computing device can transmit the user information to the POS terminal. In other implementations, the computing device transmits the user information to a POP service.

The computing device receives (S706) medication information including a name of a medication, any alternatives of the medication (including generics), and pricing. In some cases, the computing device can receive images of the medications.

The computing device displays (S708) the medication information. The example graphical user interface is shown in FIG. 5A. The consumer selects one of the options for the medication with an input (e.g., touching a touchscreen) to the computing device; and the computing device receives (S710) the input.

The computing device receives (S712) pharmacy information including names and addresses of a plurality of pharmacies. In some implementations, the plurality of pharmacies can be determined based on a geolocation of the computing device. The geolocation can be GPS coordinates or similar.

The computing device displays (S714) the names and addresses of the plurality of pharmacies, the hours of availability of the pharmacies, and payment options for the plurality of pharmacies. The computing device can filter the list of pharmacies to, e.g., those pharmacies previously assigned as favorites by the consumer, those within a predetermined distance, or a predetermined number of the closest pharmacies.

The payment options can be to prepay or pay at the pharmacy. The payment options can include an account linked to an issuer banking application or, e.g., an HSA associated with the insurer.

The computing device receives (S716) an input selecting one of the plurality of pharmacies and a payment option.

The computing device then optionally displays (S718) a pre-confirmation screen to the consumer, such as shown in FIG. 5C.

The computing device then optionally determines whether it has received an input (S720) confirming the information displayed on the pre-confirmation screen. If the computing device does not receive an input confirming the displayed information, then the computing device may return to a previous operation (e.g., S708, S714) to enable the consumer to alter the inputs. If the computing device receives an input confirming the displayed information, the computing device continues the work flow to operation S722.

The computing device transmits (S722) the selection of one of the pharmacies and a payment indication. In some cases, the computing device transmits the payment indication to the doctor POS terminal. In some cases, the computing device transmits the payment indication to the POP service.

The computing device can optionally make a payment (S724), if the consumer chose to prepay.

FIG. 7B illustrates an example workflow of a prescription optimization service performing the method illustrated in FIG. 6. Referring to FIG. 7B, the workflow begins when the POP service receives (S730) user information. The POP service also receives (S732) prescription information.

In various implementations, the POP service receives the user information and prescription information from the computing device, the doctor system, or the doctor POS terminal. The POP service can receive a portion or the entirety of the user information and the prescription information from the same device or different devices.

The POP service can determine (S734) routing to the insurance service.

The POP service obtains (S736) selection information. As discussed above, the selection information can include the alternatives, images of the alternatives, and expected pricing of the alternatives. In one implementation, the POP service transmits to the insurance service a request for the selection information. In some implementations, such as when the POP service and the insurance service are hosted on the same server, the POP service obtains the selection information from a local database.

The POP service transmits (S738) the selection information. In different implementations, the transmission can be to the doctor system, the doctor POS terminal, or the computing device.

The POP service receives (S740) the confirmed information from the doctor system, the doctor POS terminal, or the computing device.

The POP service transmits (S742) an authorization to the insurance service to dispense the medicine. The authorization can include an identifier (e.g., name) of the patient, the selected alternative, a quantity and dosage of the alternative, and an indication as to whether the consumer prepaid for the alternative.

Thus, the insurance service is informed what medication will be dispensed to the consumer. This information can prevent the consumer from obtaining too much of the same drug from different pharmacies.

If the consumer elected to prepay, then the authorization can also include reconciliation information for reconciling the financial payment from the issuer to the insurance carrier. The POP service then ends its workflow.

If the consumer elected to pay at the pharmacy, the POP service receives (S744) payment confirmation from the pharmacy. The payment information can include the name and address of the pharmacy and an amount to be paid from an account of the consumer to the pharmacy, an identifier of the patient (name, insurance carrier, and group and/or policy number). In some implementations, the payment information includes the name of the distributed medication and an amount of the medication.

The POP service reconciles (S746) the payment from the consumer to the pharmacy, based on the payment information. In some implementations, the reconciliation includes contacting the insurance service to indicate disbursement of the medicine. This contact can include transmitting from the POP service to the pharmacy any information to reconcile the payment. Such information can include all or a portion of the payment information, as well as the name of the consumer, the account number of the consumer, and the group and/or policy number of the consumer.

Modifications are possible. For example, the computing device need not transmit the user information when, for example, the doctor POS terminal or POP service receive the user information in other manners. In such a case, the doctor POS terminal or POP service can push information to the computing device. [0091.] In another implementation, the consumer enters a desired pick-up time into the computing device. The computing device communicates this information to the pharmacy. This communication can occur via the doctor POS terminal, and the doctor system, the POP service, the insurance service, or an out-of-band communication.

FIG. 8 illustrates components of an example computing device that may be used by a consumer for optimizing prescription filling. System 800 may represent a computing device such as, but not limited to, a personal computer, a reader, a mobile device, a personal digital assistant, a wearable computer, a smart phone, a tablet, or a laptop computer (notebook or netbook). As mentioned above, system 800 may embody computing device 112 and may support contactless payments.

System 800 includes a processor 805 that processes data according to instructions of operating system 820 and various applications including application 810, which includes a prescription feature as described herein. Application 810 may include instructions to perform processes such as described with respect to FIGS. 4 and 7A. Application 810 may be loaded into memory 815 and run on or in association with the operating system 820.

Processor 805 can include general purpose central processing units (CPUs), graphics processing units (GPUs), field programmable gate arrays (FPGAs), application specific processors, and logic devices, as well as any other type of processing device, combinations, or variations thereof.

Memory 815 may include volatile and nonvolatile memories, removable and non-removable media implemented in any method or technology for storage of information, such as computer readable instructions, data structures, program modules, or other data. Examples of storage media of memory 815 include random access memory, read only memory, magnetic disks, optical disks, CDs, DVDs, flash memory, magnetic cassettes, magnetic tape, magnetic disk storage or other magnetic storage devices, or any other suitable storage media. In no case does storage media consist of transitory, propagating signals.

System 800 can include a power supply 830, which may be implemented as one or more batteries and/or an energy harvester (ambient-radiation, photovoltaic, piezoelectric thermoelectric electrostatic, and the like). Power supply 830 might further include an external power source, such as an AC adapter or a powered docking cradle that supplements or recharges the batteries.

System 800 may also include a radio/network interface 835 that performs the function of transmitting and receiving communications. The radio/network interface 835 allows system 800 to communicate with other computing devices, such as over a network. The radio/network interface 835 facilitates wireless connectivity between system 800 and the “outside world,” via a communications carrier or service provider. Transmissions to and from the radio/network interface 835 are conducted under control of the operating system 820, which disseminates communications received by the radio/network interface 835 to application program 810 and vice versa. Radio/network interface 835 can further include near field communication (NFC)

An audio interface 840 can be used to provide audible signals to and receive audible signals from the user. For example, the audio interface 840 can be coupled to a speaker to provide audible output and a microphone to receive audible input, such as to facilitate a telephone conversation or voice commands.

System 800 may further include video interface 845 that enables an operation of an optional camera (not shown) to record still images, video stream, and the like. Visual output can be provided via a display 855. Visual output may be depicted on the display 855 in myriad ways, presenting graphical user interface elements, text, images, video, notifications, virtual buttons, virtual keyboards, or any other type of information capable of being depicted in visual form. In some cases, the display may be touch screen. A keypad 860 can also be included for user input. The keypad 860 may be a physical keypad or a soft keypad generated on the touch screen display 855.

Audio interface 840, video interface 845, display 855, and keypad 860 may be considered to be part of the system's user interface system, which may include input/output (I/O) devices and components that enable communication between a user and the system 800. In general, a user interface system can include input devices such as a mouse, track pad, keyboard, a touch device for receiving a touch gesture from a user, a motion input device for detecting non-touch gestures and other motions by a user, a microphone for detecting speech, and other types of input devices and their associated processing elements capable of receiving user input; and output devices such as display screen(s), speakers, haptic devices for tactile feedback, and other types of output devices. In certain cases, the input and output devices may be combined in a single device, such as a touchscreen display which both depicts images and receives touch gesture input from the user.

A user interface system may also include user interface software and associated software (e.g., for graphics chips and input devices) executed by the OS in support of the various user input and output devices. The associated software assists the OS in communicating user interface hardware events to application programs using defined mechanisms. The user interface system including user interface software may support a graphical user interface, a natural user interface, or any other type of user interface.

FIG. 9 illustrates components of an example computing system that may be used for prescription optimization. Referring to FIG. 9, system 900 can be implemented within a single computing device or distributed across multiple computing devices or sub-systems that cooperate in executing program instructions. In some implementations, the system 900 can include one or more blade server devices, standalone server devices, personal computers, routers, hubs, switches, bridges, firewall devices, intrusion detection devices, mainframe computers, network-attached storage devices, smartphones and other mobile telephones, and other types of computing devices. The system hardware can be configured according to any suitable computer architectures such as a Symmetric Multi-Processing (SMP) architecture or a Non-Uniform Memory Access (NUMA) architecture.

The system 900 can include a processor 910 that can include one or more hardware processors and/or other circuitry that retrieves and executes the software implementing the POP service 920 from storage 930. The processor 910 can be implemented within a single processing device, chip, or package but can also be distributed across multiple processing devices, chips, packages, or sub-systems that cooperate in executing program instructions.

The storage 930 can include any computer readable storage media readable by processor 910 and that stores software 920. Storage 930 can be implemented as a single storage device but can also be implemented across multiple storage devices or sub-systems co-located or distributed relative to each other. Storage 930 can include additional elements, such as a controller, that communicate with processor 910. Storage 930 can also include storage devices and/or sub-systems on which data and/or instructions are stored. System 900 can access one or more storage resources to access information to carry out any of the processes indicated by software 920.

Software 920, including routines for at least partially performing at least one of the processes for a POP service such as described with respect to FIGS. 6 and 7B can be implemented in program instructions. Further, software 920, when executed by system 900 in general or processor 910 in particular, can direct, among other functions, the system 900 or processor 910 to operate as described herein.

In implementations where the system 900 includes multiple computing devices, the server can use one or more communications networks that facilitate communication among the computing devices. For example, the one or more communications networks can include or be a local or wide area network that facilitates communication among the computing devices. One or more direct communication links can be included between the computing devices. In addition, in some cases, the computing devices can be installed at geographically distributed locations. In other cases, the multiple computing devices can be installed at a single geographic location, such as a server farm or an office.

System 900 can include a communications interface 940 that provides one or more communication connections and/or one or more devices that allow for communication between system 900 and other computing systems (not shown) over a communication network or collection of networks (not shown) or the air.

In some embodiments, system 900 can host one or more virtual machines. In some implementations, those virtual machines at least partially perform at least one of the processes carried out by a POP service as described herein.

Alternatively, or in addition, the functionality, methods and processes described herein can be implemented, at least in part, by one or more hardware modules (or logic components). For example, the hardware modules can include, but are not limited to, application-specific integrated circuit (ASIC) chips, field programmable gate arrays (FPGAs), system-on-a-chip (SoC) systems, complex programmable logic devices (CPLDs) and other programmable logic devices now known or later developed. When the hardware modules are activated, the hardware modules perform the functionality, methods and processes included within the hardware modules.

As used herein, the terms “storage media,” “computer-readable storage media,” or “computer-readable storage medium” refers to non-transitory storage media, such as a hard drive, a memory chip, and cache memory.

Although the subject matter has been described in language specific to structural features and/or acts, the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A method implemented by a computing device, comprising:
initiating an optimized prescription fill request of a medical prescription via a payment network by at least transmitting user information including a name of a patient to a payment gateway;
receiving pricing options for the medical prescription from a server;
obtaining pharmacy information of at least one pharmacy that can fill the medical prescription;
providing the pricing options via a user interface;
providing the pharmacy information of the at least one pharmacy via the user interface;
receiving a selection of a selected pharmacy of the at least one pharmacy and a payment indication; and
transmitting the selection of the selected pharmacy and the payment indication to the payment gateway.

2. The method of claim 1, further comprising:
transmitting an identification of an insurance carrier of the patient, wherein the user information includes an identification of a policy of the patient with the insurance carrier.

3. The method of claim 1, wherein the pricing options for the medical prescription comprises at least one brand and at least one generic of the medical prescription.

4. The method of claim 1, further comprising:
receiving a selection of a pricing option; and
transmitting the selection of the pricing option to the payment gateway.

5. The method of claim 1, wherein obtaining the pharmacy information comprises:
receiving a geolocation of the computing device, wherein the at least one pharmacy is based on the geolocation of the computing device.

6. The method of claim 1, wherein the user information identifies a funding primary account number or a health savings account.

7. The method of claim 1, wherein the payment indication comprises a prepayment indication for the prescription at the selected pharmacy.

8. A server supporting optimized prescription filling, comprising:
one or more processors;
one or more storage media; and
instructions stored on the one or more storage media that when executed by the one or more processors direct the server to at least:
receive a request for an optimized prescription fill, wherein the request comprises user information, including a name of a patient, and a prescription for a medicine for the patient;
obtain medication information at least including pricing information for the prescription for the medicine for the patient;
transmit the medication information to a payment application with prescription feature;
receive a selection of a selected pharmacy and a payment indication; and
transmit an authorization of a dispensation of the medicine to the patient for the selected pharmacy along with the payment indication.

9. The server of claim 8, wherein the user information further comprises an identification of a policy of the patient with an insurance carrier,
wherein the obtaining the medication information comprises transmitting a request via the insurance carrier for the medication information.

10. The server of claim 9, wherein the pricing information for the medicine comprises at least one brand and at least one generic of the medicine.

11. The server of claim 8, further comprising instructions that direct the server to:
determine routing to the payment application with prescription feature in response to receiving the request for optimized prescription fill.

12. The server of claim 8, wherein the payment indication comprises a prepayment indication for the prescription at the selected pharmacy,
wherein the instructions further direct the server to:
receive a transaction request with the prepayment indication and payment information for the prescription.

13. The server of claim 8, wherein the user information and the prescription are received from a doctor computing system via an application programming interface.

14. The server of claim 8, wherein the selected pharmacy and the payment indication are received via a payment gateway.

15. A computer-readable storage medium encoded with instructions that, when executed by a processor of a computing device, direct the computing device to:
initiate an optimized prescription fill request of a medical prescription via a payment network by at least transmitting user information including a name of a patient to a payment gateway;
receive pricing options for the medical prescription;
obtain pharmacy information of at least one pharmacy that can fill the medical prescription;
provide the pricing options via a user interface;
provide the pharmacy information of the at least one pharmacy via the user interface;
receive a selection of a selected pharmacy of the at least one pharmacy and a payment indication; and
transmit the selection of the selected pharmacy and the payment indication to the payment gateway.

16. The storage medium of claim 15, wherein the instructions further direct the computing device to:
transmit an identification of an insurance carrier of the patient, wherein the user information includes an identification of a policy of the patient with the insurance carrier.

17. The storage medium of claim 15, wherein the pricing options for the medical prescription comprises at least one brand and at least one generic of the medical prescription.

18. The storage medium of claim 15, wherein the instructions further direct the computing device to:
receive a selection of a pricing option; and
transmit the selection of the pricing option to the payment gateway.

19. The storage medium of claim 15, wherein the instructions further direct the computing device to:
receive a geolocation of the computing device, wherein the at least one pharmacy is based on the geolocation of the computing device.

20. The storage medium of claim 15, wherein the payment indication comprises a prepayment indication for the prescription at the selected pharmacy.
